(19) United States
(12) Patent Application Publication
(10) Pub. No.: US 2006/0228368 A1
(43) Pub. Date: Oct. 12, 2006

(54) Method of protecting against staphylococcal infection

(75) Inventors: Ali Fattom, Rockville, MD (US); Jawad Sarwar, Germantown, MD (US); Zuzana Kossaczka, Bethesda, MD (US); Kimberly L. Taylor, Bethesda, MD (US); Sofiane Ennifar, Silver Spring, MD (US)

Correspondence Address: ROSSI, KIMMS & McDOWELL LLP., P.O. BOX 826, ASHBURN, VA, 20146-0826, US

(73) Assignee: Nabi Biopharmaceuticals, Boca Raton, FL (US)

(21) Appl. No.: 11/101,386

(22) Filed: Apr. 7, 2005

(51) Int. Cl. A61K 39/40; A61K 39/085

(52) U.S. Cl. 424/164.1; 424/243.1

(57) ABSTRACT

A method of preventing or treating staphylococcal bacterial infection in an individual is disclosed. A vaccine based on a conjugate the 336 polysaccharide antigen can be used for active protection in individuals who are to be subjected to conditions that place them at immediate risk of developing a bacterial infection, as would be case in the context of a catheterization or a surgical procedure. Alternatively, antibodies raised in response to the antigen can be used to treat or to provide passive protection to individuals. The method can be used in a population of patients at risk for infection by various species of Staphylococcus or various types of Staphylococcus aureus.

### BACKGROUND OF THE INVENTION

A. Field of the Invention

The invention relates generally to the use of staphylococcal vaccines in preventing bacterial infection in an individual.

B. Description of the Related Art

Staphylococci and Enterococci rarely cause systemic infections in otherwise healthy individuals, and therefore are considered opportunistic pathogens. Through various mechanisms, normal adult humans and animals with a competent immune system attain an innate natural resistance to these bacterial infections. These include mucosal and epidermal barriers, in addition to possible immunological mechanisms. Interruption of these natural barriers as a result of injuries such as burns, traumas, or surgical procedures involving indwelling medical devices, increases the risk for staphylococcal and enterococcal infections. In addition, individuals with a compromised immune response such as cancer patients undergoing chemotherapy and radiation therapy, diabetes, AIDS, alcoholics, drug abuse patients, post organ transplantation patients and infants are at an increased risk for staphylococcal and enterococcal infections.

Staphylococci are commensal bacteria of the anterior nares, skin, and the gastrointestinal tract of humans. It is estimated that staphylococcal infections account for >50% of all hospital acquired infections. S. aureus alone is responsible for 15-25% of such infections and is surpassed only by S. epidermidis, which accounts for 35% of these infections. Staphylococcal infections, especially those caused by S. aureus are associated with high morbidity and mortality.

Staphylococcus and Enterococcus are a major cause of nosocomial and community-acquired infections, including bacteremia, metastatic abscesses, septic arthritis, endocarditis, osteomyelitis, and wound infections. For example, the bacteremia-associated overall mortality for S. aureus is approximately 25 percent. A study of hospitalized patients in 1995 found that death rate, length of stay, and medical costs were twice as high for S. aureus-associated hospitalizations compared with other hospitalizations. S. aureus bacteremia is a prominent cause of morbidity and mortality in hemodialysis patients with an annual incidence of three to four percent. Contributing to the seriousness of S. aureus infections is the increasing percentage of isolates resistant to methicillin, and early reports of resistance to vancomycin. Hence, immunoprophylaxis against S. aureus is highly desired.

The capsular polysaccharides (CPS) of S. aureus are virulence factors in systemic infections caused by this opportunistic pathogen. S. aureus CPS confer invasiveness by inhibiting opsonophagocytic killing by polymorphonuclear neutrophils (PMN), similar to other encapsulated bacteria, such as Streptococcus pneumoniae. This enables the bacteria to persist in the blood, where they elaborate several different virulence factors, including toxins and extracellular enzymes. Of the 13 known types of S. aureus, Types 5 and 8 account for approximately 88 percent of all clinical isolates. Nearly all of the remaining isolates are of Type 336 that carries a more recently identified polysaccharide (PS) antigen known as 336PS. Antibodies to Types 5 and 8 capsular polysaccharides (“T5CPS” and “T8CPS”) and 336PS induce type-specific opsonophagocytic killing by human PMNs in vitro, and confer protection against the homologous strain in animal infection models.

S. aureus causes several diseases by various pathogenic mechanisms. The most frequent and serious of these diseases are bacteremia and its complications in hospitalized patients. In particular, S. aureus can cause wound infections and infections associated with catheters and prosthetic devices. Serious infections associated with S. aureus bacteremia include osteomyelitis, invasive endocarditis and septicemia. Staphylococci have developed very sophisticated mechanisms for inducing diseases in humans, including both intracellular and extracellular factors. For instance, S. aureus possesses other surface antigens that facilitate its survival in the blood stream by helping the bacteria to evade phagocytic killing by the host leukocytes. These surface antigens include cell wall components such as teichoic acid, protein A, and capsular polysaccharides (CPS). Due in part to the versatility of these bacteria and their ability to produce extracellular products that enhance virulence and pathogenicity, staphylococcal bacteremia and its complications continue to be serious and frequently observed nosocomial infections.

Antibiotics such as penicillin have been used successfully against both staphylococcal and enterococcal infections in humans, but more recently the effectiveness of such antibiotics has been thwarted by the ability of bacteria to develop resistance. For example, shortly after the introduction of methicillin, the first semisynthetic penicillin, strains of methicillin-resistant S. aureus (MRSA) were isolated. Antibiotic resistance among staphylococcal isolates from nosocomial infections continues to increase in frequency, and resistant S. aureus strains continue to cause epidemics in hospitals in spite of developed preventive procedures and extensive research into bacterial epidemiology and antibiotic development. Enterococci resistant to vancomycin started to appear in 1988 and have now become commonplace among hospital-acquired infections. Although methicillin-resistant S. aureus organisms with intermediate resistance to vancomycin have been identified in some centers, it was only recently that three S. aureus strains with complete resistance to vancomycin were reported. This suggests that the probable conjugal transfer of vancomycin resistance from Enterococci to Staphylococci has become a reality, and dissemination of these strains could eventually lead to the widespread development of organisms that are more difficult to eradicate. The problem is compounded by multiple antibiotic resistance in hospital strains, which severely limits the choice of therapy.

The initial efficacy of antibiotics in treating and curing staphylococcal infections drew attention away from immunological approaches for dealing with these infections. Although multiple antibiotic-resistant strains of S. aureus have emerged, other strategies such as vaccines have not been developed. In addition, passive immunization has been tested for use in immune-compromised individuals, such as neonates, who are at increased risk for contracting these bacterial infections. The data failed to support a solid conclusion in recommending the use of passive immunization in this population. Baker et al., New Engl. J. Med. 35:213-219 (1992); Fanaroff et al., New Engl. J. Med. 330:1107-1113 (1994).

While polysaccharide vaccines have been developed for some primary bacterial pathogens that induce acute diseases in normal individuals, namely, Streptococcus pneumoniae, Neisseria meningitidis and Hemophilus influenzae, prior to development of StaphVAX® (Nabi Biopharmaceuticals, Rockville, Md.), none had been described specifically for protection against opportunistic bacteria. This vaccine against S. aureus infections is currently in a confirmatory Phase III clinical trial in end-stage renal (kidney) disease patients.

StaphVAX® is a conjugate vaccine against two serotypes of S. aureus: Type 5 and Type 8. In the 1980s, eight different serotypes of S. aureus were identified using polyclonal and monoclonal antibodies to capsular polysaccharide (CPS). Karakawa et al., J. Clin. Microbiol. 22:445 (1985). (The contents of this document and all others listed herein are incorporated herein by reference.) Surveys have shown that approximately 85% of isolates are capsular polysaccharide Type 5 or Type 8. More recently, Nabi Biopharmaceuticals has identified and patented an antigen, 336PS, which is found on newly discovered serotype Type 336 of Staphylococcus aureus. This serotype accounts for approximately 10-12 percent of all clinically significant S. aureus infections. In the present context, a “clinically-significant” bacterial strain is one that is pathogenic in humans. The antigen was identified, purified and characterized, and a prototype conjugate vaccine based on the antigen demonstrated the ability to protect animals from challenge with clinical isolates of the homologous serotype. Nabi Biopharmaceuticals is developing a second generation of StaphVAX® vaccine that will contain 336PS antigen in addition to S. aureus Types 5 and 8 antigens. These second-generation vaccines are expected to provide coverage for nearly 100% of all clinically significant S. aureus infections.

In addition to S. aureus, Staphylococcus epidermidis is another clinically significant Gram-positive bacterium that causes hospital-acquired infections. S. epidermidis Conjugate Vaccine is an investigational vaccine in preclinical development for the prevention of S. epidermidis infections. This vaccine has been shown to induce antibodies that are protective in animal models and facilitate elimination of bacteria by the same type of immune system response as StaphVAX®. To date, none of these vaccines has been shown to provide protection against non-homologous strains of bacteria.

Nabi's S. aureus vaccine provides a solution for the problem of antibiotic resistance in Type 5 and Type 8 strains, and proposed next-generation vaccines address the same issue for other strains. However, there was no reason to expect that a vaccine based on 336PS would be effective in protecting individuals against infection by non-homologous strains of bacteria.

### SUMMARY OF THE INVENTION

The present inventors have found that conjugates of 336PS are effective in protecting against bacterial infection by strains of bacteria other than those that are classified as Type 336 when serotyped. More particularly, a conjugate vaccine comprising 336PS confers protection against infection by other S. aureus strains and against S. epidermidis. In particular, it confers protection against infection by both Type 336/5 and Type 336/8 strains of S. aureus that are described herein, as well as infection by S. epidermidis. This was entirely unexpected as it was not known that conjugates of 336PS could stimulate the production of antibodies that combat bacterial infection by strains other than Type 336 strains. Absent such a teaching, the scope of protection offered by 336PS conjugate vaccines could not have been expected.

Based on the inventors' discovery, a method now is provided for preventing infection in a population of patients at risk for infection by various species of Staphylococcus or various types of Staphylococcus aureus, comprising administering to a patient in the population a composition comprising a conjugate of an isolated S. aureus antigen that contains N-acetylglucosamime linked to ribitol, wherein the antigen binds with antibodies to S. aureus Type 336 deposited under ATCC 55804. The conjugate of the isolated S. aureus antigen produces antibodies that protect against the homologous serotype and species or serotype of Staphylococcus other than S. aureus Type 336. The present invention further provides a method for preventing infection in a population of patients at risk for infection by Staphylococcus epidermidis, comprising administering to a patient in the population a composition comprising a conjugate of an isolated S. aureus antigen that contains N-acetylglucosamime linked to ribitol, wherein the antigen binds with antibodies to S. aureus Type 336 deposited under ATCC 55804. Conjugates of the isolated S. aureus antigen produce antibodies that protect against S. epidermidis. The antigen comprises a 1,5-poly(ribitol phosphate) polymer chain in which the 3-position of the ribitol is substituted by N-acetyl-β-D-glucosaminyl residues.

Also provided is a method for treating infection in a population of patients at risk for developing infection by various species of Staphylococcus or various types of Staphylococcus aureus, comprising administering to a patient in the population a composition comprising antibodies to a conjugate of an isolated S. aureus antigen that contains N-acetylglucosamime linked to ribitol, wherein the antigen binds with antibodies to S. aureus Type 336 deposited under ATCC 55804. The conjugate of the isolated S. aureus antigen produces antibodies that protect against various species of Staphylococcus or various types of S. aureus other than Type 336. The present invention also provides a method for treating infection in a patient diagnosed as having a S. epidermidis infection, comprising administering to the patient a composition comprising antibodies to a conjugate of an isolated S. aureus antigen that contains N-acetylglucosamime linked to ribitol, wherein the antigen binds with antibodies to S. aureus Type 336 deposited under ATCC 55804.

### BRIEF DESCRIPTION OF THE DRAWINGS

The foregoing advantages and features of the invention will become apparent upon reference to the following detailed description and the accompanying drawings, of which:

FIG. 1 is a pie chart that shows the distribution of surface and capsular polysaccharide serotypes of 234 S. aureus clinical isolates from bacteremic patients

FIG. 2 is a bar graph demonstrating opsonic killing of mixed serotype S. aureus isolates by purified 336PS specific 336 conjugate rabbit IgG (“336-IgG”).

FIG. 3 is a bar graph of S. epidermidis bacteremia clearance in a mouse model.

### DETAILED DESCRIPTION OF SPECIFIC EMBODIMENTS

It surprisingly has been discovered that vaccines based on conjugates of 336PS can effectively protect individuals against bacterial infection not only by homologous strains of bacteria that type as S. aureus 336 strains, but also by strains of S. aureus that type as other than Type 336, as well as by strains of S. epidermidis. There are very few polysaccharide-based vaccines that provide protection against bacterial infection, and protection against non-homologous strains of bacteria has not been reported for any of these. Accordingly, it was quite surprising to discover that a conjugate vaccine based on antigen isolated from the 336 serotype of S. aureus provided protection against some non-homologous Type 5 and Type 8 strains of S. aureus and against strains of S. epidermidis.

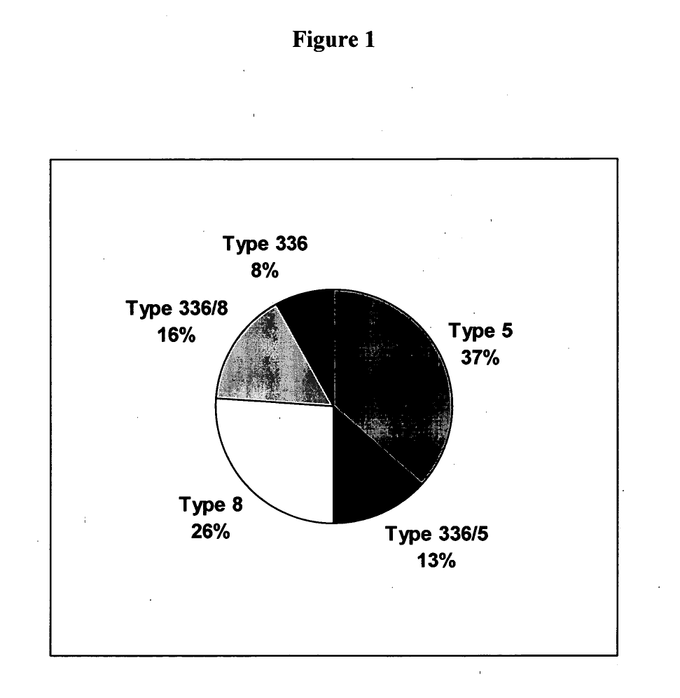

It appears that in some strains that type as Type 5 and Type 8 S. aureus, the capsule is discontinuous which allows exposure of an antigen that is serologically cross-reactive with antibodies that are raised against 336PS conjugate (336PS covalently bound to protein) vaccine. These strains therefore type serologically as both Type 336 and one of Type 5 or Type 8. They are denoted herein as “mixed Type 336/5” and “mixed Type 336/8,” and account for approximately 29% of clinically significant isolates. The distribution of surface and capsular polysaccharide serotypes of 234 S. aureus clinical isolates from bacteremic patients is shown in FIG. 1. The isolates were serotyped using antibodies generated by immunizations of rabbits with Type 5, Type 8 or Type 336 polysaccharide conjugated to Pseudomonas aeruginosa exoprotein A (rEPA). The 336 phenotype was found to be present on 37% of all the clinical isolates, which include 8% 336, 13% 336/5, and 16% 336/8. As a surprising correlate of this discovery, it has been shown that IgG generated in response to a 336PS conjugate vaccine is able to mediate opsonophagocytosis of serotype 336, 336/5 and 336/8 strains.

Quite unexpectedly, antibodies generated in response to a 336PS conjugate vaccine also possess the ability to protect against infections in which S. epidennidis is the causative organism. IgG derived from 336PS conjugate vaccine shows cross-reactivity with a S. epidermididis polysaccharide antigen that is found on clinical isolates. Furthermore, immunoglobulin raised in response to 336PS conjugate vaccine efficiently cross-clears S. epidermidis bacteremia in a mouse model.

Antigen for the preparation of a conjugate vaccine according to the present invention is described in U.S. Pat. No. 5,770,208, the contents of which are incorporated by reference in their entirety. This patent describes that virtually all clinical isolates strains of S. aureus that do not serotype as Type 5 or Type 8 serotype as Type 336. In U.S. Pat. No. 5,770,208, the “336PS antigen” is combined with Type 5 and Type 8 CPS antigens, to produce a vaccine that provides almost complete protection against infection by clinically significant S. aureus isolates. In this regard, a “clinically significant” isolate is an isolate that is pathogenic. More particularly, typing of isolates obtained from various sources has shown that approximately 60% of isolates are Type 8, approximately 30% are Type 5 and that nearly all of the remaining 10% of isolates are Type 336. Less than 1% of the isolates do not type as one of these three types.

U.S. Pat. No. 5,770,208 reports that antibodies to S. aureus 336 do not cross-react with serotype specific polysaccharides isolated from any of S. aureus Type 5, Type 8, Type 4, K73 (a Type 5 variant strain) or S. epidermidis. The antibodies in this case were a whole cell antiserum raised against Type 336 cells. The results for whole cell antiserum contrast with the results obtained with antiserum derived from 336PS conjugate (336PS covalently bound to protein), as the latter do react with Type 336/5, Type 336/8 and S. epidermidis. Indeed, immunodiffusion studies demonstrate a broad reactivity of 336PS conjugate antiserum, e.g., towards S. aureus 336PS, S. aureus teichoic acid (SA TA) and S. epidermidis PS1. In contrast, immunodiffusion studies with anti-336 whole cell serum demonstrate a specificity towards 336PS, i.e., 336PS isolated from a Type 336 isolate gives a positive reaction with homologous type whole cell antiserum by immunodiffusion test and therefore was stated to be type-specific. It is postulated that conjugation of 336PS in the form of conjugate with protein induces significant amounts of antibodies that recognize epitopes not only on Type 336 cells, but also epitopes on Type 336/5, Type 336/8 and S. epidermidis.

The antigen can be obtained in recoverable amounts, from certain S. aureus isolates cultured pursuant to the protocols described herein, in substantially pure form. In particular, purified antigen acceptable for human use contains minimal amounts of other materials such as proteins and nucleic acids, and is of vaccine-grade quality as defined by the FDA. A “recoverable” amount in this regard means that the isolated amount of the antigen is detectable by a methodology less sensitive than radiolabeling, such as immunoassay, and can be subjected to further manipulations involving transfer of the antigen per se into solution.

To obtain 336PS, a 336 isolate according to the invention can be grown, for example, in Columbia Broth supplemented with 2% NaCl, although other media can be substituted. Following fermentation, cells are killed, and then harvested by centrifugation. Antigen preferably is extracted from cell paste.

Enzyme treatments of cell paste with lysostaphin, DNase, RNase and optionally protease, followed by sequential precipitation with 25-75% cold ethanol/CaCl₂, results in a crude antigen extract. The crude antigen extract is treated with lysozyme and purified by size on a suitable size exclusion matrix and the 336PS positive fractions are then pooled, concentrated, dialyzed and lyophilized. The lyophilized material is dissolved in buffer and loaded onto an ion-exchange column equilibrated with the same buffer. The column is washed with NaCl loading buffer and then eluted with a NaCl gradient. Fractions containing antigen are pooled, dialyzed, concentrated, and lyophilized. The separation can be repeated to obtain better purification. The foregoing protocol is exemplary; various protocols can be followed to extract and purify 336PS in accordance with the present invention.

Analysis of purified 336PS shows that it comprises N-acetyl glucosamine and ribitol. The antigen comprises a 1,5-poly(ribitol phosphate) polymer chain in which the 3-position of the ribitol is substituted by N-acetyl-β-D-glucosaminyl residues.

This structure is distinct from that of the S. aureus poly(ribitol phosphate)teichoic acid where the N-acetyl-β-D-glucosaminyl residues are attached to the 4-position of the ribitol.

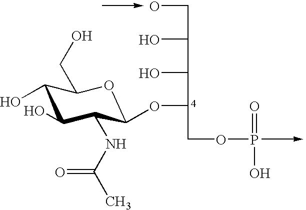

Although 336PS is by chemical composition similar to S. aureus teichoic acid, structurally it is different. What appear to be slight differences in their primary structure, i.e., GlcNAc binds in the C3 of ribitol instead of C4 of ribitol in a polymer, apparently results in dramatically different effects of periodate oxidation on these compounds. The structural difference likely also accounts for the differences in serological reactivities. The seemingly slight difference in primary structure might have considerable consequences in terms of folding of the polymer and epitope configuration and conformation, leading to the distinctiveness of the antigen by serologic tests, e.g., Ouchterlony assay, ELISA and inhibition ELISA.

The antigen also is chemically distinct from both the Type 5 and Type 8 S. aureus antigens. The structures of Types 5 and 8 polysaccharide antigens have been elucidated by Moreau et al., Carbohydr. Res. 201:285 (1990); and Fournier et al., Infect. Imm. 45:87 (1984). Both have N-acetylfucosamine in their repeat unit as well as N-acetylmannosamine. Their structures were reported as:
→4)-β-D-ManpNAcA(3OAc)-(1→4)-α-L-FucpNAc-(1→3)-β-D-FucpNAc-(1-> Type 5:
→3)-β-D-ManpNAcA(4OAc)-(1→3)-α-L-FucpNAc-(1→3)-β-D-FucpNAc-(1-> Type 8:

Induction of bacteremia in mammals requires extremely high numbers of organisms or some previous maneuver to lower the host resistance. In vitro phagocytosis mediated by specific antibodies to bacterial polysaccharide, however, can be used as a correlate of protective immunity in vivo. In this model, the ability of 336PS-specific monoclonal and polyclonal antibodies to opsonize S. aureus in vitro is measured by phagocytosis, according to the method described in Kojima et al., Infect Dis. Immun. 58: 2367-2374 (1990).

As reported in U.S. Pat. No. 5,770,208, antibodies induced by a type 336PS vaccine facilitate type-specific phagocytosis, and it was also reported that the in vitro phagocytosis assays indicated that antibodies to 336PS are protective against infection by S. aureus strains that carry 336PS. There was no suggestion that antibodies to the conjugate of 336PS would be protective against S. aureus strains that react serologically with antiserum raised against Type 5 or Type 8 strains.

Preferably, a composition of the antigen/immunocarrier conjugate according to the present invention “consists essentially of” the conjugate. In this context, the phrase “consists essentially of” means that the composition does not contain any material that negatively impacts the elicitation of an immune response to the antigen (and to other antigens, if present) when the composition is administered to a subject as a vaccine. Preferably the composition does not contain a substantial amount of unconjugated antigen.

Bacterial capsular polysaccharides are generally poor immunogens. Polysaccharide antigens normally generate a T-cell independent immune response and they induce humoral antibodies with no boost of the immune response observed upon reinjection. To generate a complete immune response, conjugation of polysaccharide to protein carriers can alter bacterial CPS antigens to make them T-cell dependent immunogens, thus increasing their immunogenicity and potentiating their use in infants and immune-compromised patients. Therefore, for use in a vaccine, it is preferable to conjugate the antigen to an immunocarrier, usually a polypeptide or protein, thereby to improve qualitatively and quantitatively the host humoral immune response specific to the PS antigen by recruiting T cells and interaction between T and B cells for the induction of an immune response against the PS antigen. This is particularly important for vaccines intended for use in patients with reduced resistance.

An immunocarrier thus enhances immunogenicity both for active immunization and for preparing high-titered antisera in volunteers for passive immunization. Suitable immunocarriers according to the present invention include tetanus toxoid, diphtheria toxoid, Pseudomonas aeruginosa Exotoxin A or its derivatives, recombinantly-produced non-toxic mutants of exotoxin A, as described, for example, in Fattom et al., Inf and Imm. 61: 1023-1032 (1993), as well as other proteins commonly used as immunocarriers.

Hydroxyl groups on the antigen can be activated using cyanogen bromide or 1-cyano4-dimethylamino-pyridinium tetrafluoroborate and bound, through a linker containing nucleophilic group(s) or without a linker, to a suitable immunocarrier such as a protein, e.g., diphtheria toxoid (DTd), recombinant exoprotein A from Pseudomonas aeruginosa (rEPA), or tetanus toxoid (TTd). See, for example, Kohn et al. FEBS Left. 154: 209:210 (1993); Schneerson, et al., J. Exp. Med 152:361-376 (1980); Chu et al. Infect. Immun. 40:245-256 (1983); Kossaczka, et al., Infect Immun. 68:5037-5043 (2000). The resulting conjugates are separated from unconjugated antigen.

There are other conjugation methods known in the art, e.g., periodate oxidation followed with reductive amination, carbodiimide treatment, and other methods and/or their different combinations that can provide direct or indirect (through a linker) covalent binding of 336PS and carrier protein and thus yield the conjugate. Regardless of the method used to conjugate the antigen to the carrier protein, the covalent binding of 336PS to carrier protein converts 336PS from a T cell independent antigen to a T cell dependent antigen. As a result, 336PS-protein conjugate elicited 336PS-specific antibody response in immunized animals in contrast to no such response observed upon administering 336PS alone.

Preferably the conjugate is administered without an adjuvant in order to avoid adjuvant-induced toxicity. If an adjuvant is used, it is preferred to use one that promotes humoral immune response and is acceptable for human use, e.g., aluminum hydroxide, aluminum phosphate, QS-21. Efficient adjuvants to be used experimentally include complete Freund's adjuvant (CFA) and incomplete Freund's adjuvant (IFA). A vaccine according the invention additionally may comprise a yeast or a fungal derived β-glucan or its derivatives, in particular, a baker yeast β-glucan as described in U.S. Pat. No. 6,355,625.

The 336PS conjugate according to the present invention is the active ingredient in a composition, which additionally may comprise a pharmaceutically acceptable excipient for the active ingredient. In this regard, a pharmaceutically acceptable excipient is a material that can be used as a vehicle for administering a medicament because the material is inert or otherwise medically acceptable, as well as compatible with the active agent, in the context of vaccine administration. In addition to a suitable excipient, the composition can contain conventional vaccine additives like diluents, adjuvants, antioxidants, preservatives and solubilizing agents. The vaccine can induce production in vivo of antibodies that combat S. aureus infection.

The present invention is particularly based on the ability of antibodies specific to 336PS, that are elicited in response to 336PS conjugate, to mediate protection against not only homologous strains of bacteria but also against heterologous strains. This results from the heretofore unrealized cross-reactive capacity of antibodies elicited by 336PS conjugate to other surface polysaccharides of other staphylococcal species, strains and serotypes.

The present invention also relates to the use of the 336PS conjugate to produce polyclonal antibodies or monoclonal antibodies (mouse or human) that bind to S. aureus strains that carry 336PS and/or antigens that cross-react with antibodies to 336PS, thereby mediating their clearance. Protocols for producing these antibodies are described in Ausubel, et al. (eds.), Molecular Cloning: A Laboratory Manual, Cold Spring Harbor Laboratory, Cold Spring Harbor, N.Y.)., Chapter 11; in METHODS OF HYBRIDOMA FORMATION 257-271, Bartal & Hirshaut (eds.), Humana Press, Clifton, N.J. (1988); in Vitetta et al., Immunol. Rev. 62:159-83 (1982); and in Raso, Immunol. Rev. 62:93-117 (1982).

Inoculum for polyclonal antibody production typically is prepared by dispersing the conjugate in a physiologically-tolerable diluent such as phosphate buffered saline (PBS). An immunostimulatory amount of inoculum, with or without adjuvant, is administered to a mammal and the inoculated mammal is then maintained for a time period sufficient for the antigen to induce protecting 336PS specific antibodies. Boosting doses of the conjugate may be used in individuals that are not already primed to respond to the antigen.

Antibodies can include antibody preparations from a variety of commonly used animals, e.g., goats, primates, donkeys, swine, rabbits, horses, hens, guinea pigs, rats, and mice, and even human antibodies after appropriate selection, fractionation and purification. Animal antisera may also be raised by inoculating the animals with formalin-killed 336 strains of S. aureus, by conventional methods, bleeding the animals and recovering serum or plasma for further processing.

The antibodies induced in this fashion can be harvested and isolated to the extent desired by well known techniques, such as by alcohol fractionation and column chromatography, or by immunoaffinity chromatography; that is, by binding antigen to a chromatographic column, passing the antiserum through the column, thereby retaining specific antibodies and separating out other immunoglobulins (IgGs) and contaminants, and then recovering purified antibodies by elution with a chaotropic agent, optionally followed by further purification, for example, by passage through a column of bound blood group antigens or other non-pathogen species. This procedure may be preferred when isolating the desired antibodies from the sera or plasma of humans that have developed an antibody titer against the pathogen in question, thus assuring the retention of antibodies that are capable of binding to the antigen. They can then be used in preparations for passive immunization against 336 strains of S. aureus as well as against heterologous strains of S. aureus, and even against other species of Staphylococcus.

A monoclonal 336PS specific antibody composition contains, within detectable limits, only one antibody specificity capable of binding to an epitope on 336PS or an epitope of a cross-reactive antigen. Suitable antibodies in monoclonal form can be prepared using conventional hybridoma technology.

To form hybridomas from which a monoclonal antibody composition of the present invention is produced, a myeloma or other self-perpetuating cell line is fused with lymphocytes obtained from peripheral blood, lymph nodes or the spleen of a mammal hyperimmunized with 336PS conjugate. It is preferred that the myeloma cell line be from the same species as the lymphocytes. Splenocytes are typically fused with myeloma cells using polyethylene glycol 1500. Fused hybrids are selected by their sensitivity to HAT. Hybridomas secreting the antibody molecules of this invention can be identified using an ELISA.

A BALB/c mouse spleen, human peripheral blood, lymph nodes or splenocytes are the preferred materials for use in preparing murine or human hybridomas. Suitable mouse myelomas for use in the present invention include the hypoxanthine-aminopterin-thymidine-sensitive (HAT) cell lines, a preferred myeloma being P3X63-Ag8.653. The preferred fusion partner for human monoclonal antibody production is SHM-D33, a heteromyeloma available from ATCC, Manassas, Va. under the designation CRL 1668.

A monoclonal antibody composition of the present invention can be produced by initiating a monoclonal hybridoma culture comprising a nutrient medium containing a hybridoma that secretes antibody molecules of the appropriate specificity. The culture is maintained under conditions and for a time period sufficient for the hybridoma to secrete the antibody molecules into the medium. The antibody-containing medium is then collected. The antibody molecules then can be isolated further by well known techniques.

Media useful for the preparation of these compositions are both well known in the art and commercially available, and include synthetic culture media, inbred mice and the like. An exemplary synthetic medium is Dulbecco's Minimal essential medium supplemented with 20% fetal calf serum. An exemplary inbred mouse strain is the BALB/c.

Other methods of preparing monoclonal antibody compositions are also contemplated, such as interspecies fusions, since it is primarily the antigen specificity of the antibodies that affects their utility in the present invention. Human lymphocytes obtained from infected individuals can be fused with a human myeloma cell line to produce hybridomas that can be screened for the production of antibodies that recognize 336PS. More preferable in this regard, however, is a process that does not entail the use of a biological sample from an infected human subject. For example, a subject immunized with a vaccine as described herein can serve as a source for antibodies suitably used in an antibody composition within the present invention.

In a particularly preferred embodiment, monoclonal antibodies are produced to 336PS using methods similar to those described for type-specific antibodies to S. aureus Type 5 and Type 8. The purified monoclonal antibodies are characterized by bacterial agglutination assays using a collection of clinical isolates.

The monoclonal and polyclonal antibody compositions produced according to the present description can be used in passive immunization to introduce antibodies that mediate opsonophagocytosis for the treatment of infection by strains of Staphylococcus that carry 336PS and/or an antigen that cross-reacts with antibodies raised to 336PS conjugate. Such strains include, but are not necessarily limited to, Type336/5, Type 336/8 and S. epidermidis. In this regard, the antibody preparation can be a polyclonal composition. Such a polyclonal composition may include, in addition to the antibodies that bind to 336PS and/or antigens that cross-react with antibodies raised to the 336PS conjugate, antibodies that bind to the antigens that characterize Type 5 and Type 8 strains of S. aureus. Such a composition can be obtained by immunizing a population with a multivalent vaccine or by mixing antibodies raised in separate populations in response to univalent vaccines. Thus, the polyclonal antibody component can be a polyclonal antiserum, preferably affinity purified, from an animal that has been immunized with the 336PS conjugate, and preferably also with Type 5 and Type 8 antigen conjugates. Alternatively, an “engineered oligoclonal” mixture may be used, such as a mixture of monoclonal antibodies to 336PS, and monoclonal antibodies to the Type 5 and/or Type 8 antigens.

In both types of mixtures, it can be advantageous to link antibodies together chemically to form a single polyspecific molecule capable of binding to 336PS or to a cross-reactive antigen, and to one or both of Type 5 and Type 8 antigens. One way of effecting such a linkage is to make bivalent F(ab′)₂ hybrid fragments by mixing two different F(ab′)₂ fragments produced, e.g., by pepsin digestion of two different antibodies, reductive cleavage to form a mixture of Fab′ fragments, followed by oxidative reformation of the disulfide linkages to produce a mixture of F(ab′)₂ fragments including hybrid fragments containing a Fab′ portion specific to each of the original antigens. Methods of preparing such hybrid antibody fragments are disclosed in Feteanu, Labeled Antibodies In Biology And Medicine 321-23, McGraw-Hill Int'l Book Co. (1978); Nisonoff, et al., Arch Biochem. Biophys. 93: 470 (1961); and Hammerling, et al., J Exp. Med. 128:1461 (1968); and in U.S. Pat. No. 4,331,647.

Other methods are known in the art to make bivalent fragments that are entirely heterospecific, e.g., use of bifunctional linkers to join cleaved fragments. Recombinant molecules are known that incorporate the light and heavy chains of an antibody, e.g., according to the method of Boss et al., U.S. Pat. No. 4,816,397. Analogous methods of producing recombinant or synthetic binding molecules having the characteristics of antibodies are included in the present invention. More than two different monospecific antibodies or antibody fragments can be linked using various linkers known in the art.

An antibody component produced in accordance with the present invention can include whole antibodies, antibody fragments, or subfragments. Antibodies can be whole immunoglobulin of any class, e.g., IgG, IgM, IgA, IgD, IgE, chimeric antibodies or hybrid antibodies with dual or multiple antigen or epitope specificities, or fragments, e.g., F(ab′)₂, Fab′, Fab and the like, including hybrid fragments, and additionally includes any immunoglobulin or any natural, synthetic or genetically engineered protein that acts like an antibody by binding to a specific antigen to form a complex. In particular, Fab molecules can be expressed and assembled in a genetically transformed host like E. coli. A lambda vector system is available thus to express a population of Fab's with a potential diversity equal to or exceeding that of subject generating the predecessor antibody. See Huse, W. D. et al., Science 246: 1275-81 (1989).

The present invention comprehends the protecting of a human at risk for infection by various species of Staphylococcus or various types of Staphylococcus aureus. The method comprises administering to a patient in such a population a composition comprising a conjugate of 336PS. The 336PS conjugate produces antibodies that protect against a species or type of Staphylococcus other than S. aureus Type 336. The vaccine is administered in a dose that produces a serotype-specific antibody level in the individual that is sufficient to provide immunity against challenge.

The method can be used to protect against bacterial infection in immune-compromised individuals, and produces in immune-compromised individuals a level of serotype-specific antibody to the antigens contained in the vaccines that is the same, within the limits of expected experimental variation, to the level that is achieved in normal healthy subjects when they are immunized. This was entirely unexpected in light of conventional theory to the effect that immune-compromised individuals cannot be expected to mount an effective immune response against poorly immunogenic antigens such as polysaccharide antigens, which are known for their generally low immunogenicity. There are a large number of immune-compromised populations that benefit from the administration of vaccines according to the present invention. Immune-compromised individuals include end stage renal disease (ESRD) patients; cancer patients on immunosuppressive therapy, AIDS patients, diabetic patients, the elderly in extended care facilities, patients with autoimmune disease on immunosuppressive therapy, transplant patients, and burn patients.

Preferably the 336PS-conjugate vaccine or adjuvanted vaccine is formulated to contain a target dose of at least about 5 μg of Type 336PS and up to about 500 μg of Type 336PS. Preferably at least 25 μg of Type 336PS, and more preferably 50, 75 or 100 μg of Type 336PS is used. A higher initial dose and/or a second dose of the vaccine given after the first dose may be used, particularly in immune-compromised populations because of the anticipated weaker immune response in this chronically-ill population. The vaccine provides a concentration of antibody of at least 15-20 μg/mL and a level that is at least two fold greater, and preferably four fold greater, than the prevaccination level.

The vaccine can be used for active protection in immune-compromised individuals that are about to be subjected to conditions that place them at immediate risk of developing a bacterial infection. These conditions would include, for example, catheterization or a surgical procedure. Notably, the present inventors found that even immune-compromised individuals mounted an effective immune response when vaccinated with a vaccine according to the present invention.

Pursuant to the present invention, such a vaccine can be administered to a subject not already infected with Staphylococcus, thereby to induce a staphylococcal-protective immune response in that subject. Alternatively, a vaccine within the present invention can be administered to a subject in whom staphylococcal infection already has occurred but is at a sufficiently early stage that the immune response produced to the vaccine effectively inhibits further spread of infection. Notably, the 336PS conjugate vaccine can prevent bacteremia from developing.

By another approach, a vaccine of the present invention can be administered to a subject who then acts as a source for globulin, produced in response to challenge from the specific vaccine (“hyperimmune globulin”), that contains antibodies directed against S. aureus. A subject thus treated would donate plasma from which hyperimmune globulin would then be obtained, via conventional plasma-fractionation methodology, and administered to another subject in order to impart resistance against or to treat staphylococcal infection. Hyperimmune globulins according to the invention are particularly useful for immune-compromised individuals, for individuals undergoing invasive procedures or where time does not permit the individual to produce his own antibodies in response to vaccination.

Similarly, monoclonal or polyclonal antibodies to 336PS of S. aureus produced according to the present invention can be conjugated to an immunotoxin, and administered to a subject in whom S. aureus infection has already occurred but has not become widely spread. To this end, antibody material produced pursuant to the present description would be administered in a pharmaceutically acceptable carrier, as defined herein.

The present invention is further described by reference to the following, illustrative examples.

### EXAMPLE 1

### Fermentation of S. aureus

A S. aureus 336 isolate according to the invention first is grown on a Columbia Broth agar plate supplemented with 2% MgCl₂ and 0.5% CaCl₂. A single colony is inoculated into starter culture of Columbia broth containing 2% NaCl and grown overnight with shaking at 37° C. The cells are grown in a 50-liter fermentor that contains the same medium and fermented at 37° C with agitation at 200 rpm for 24 hours, to an A_{650 nm} of 20.0.

Cells for purification of antigen were killed by adding phenol-ethanol (1:1, vol/vol) to the fermentor to a final concentration of 2%, and mixing slowly for 2 hours at 15-20° C. No viable cells were detected after this treatment. The cells then were harvested by centrifugation at 14,500×g and stored at −70° C. until use. Approximately 800-900 grams of cell paste (net weight) were obtained from a 50-liter fermentation.

### EXAMPLE 2

### Purification of Antigen

The cell paste was suspended at 0.5 g (wet weight) per ml in 0.05 M Tris-2 mM MgSo.sub.4, pH 7.5. Lysostaphin (100 to 150 μg/ml) was added and incubated at 37° C. for 3 hours with mixing. Thereafter, DNase and RNase were added to final concentrations of 50 μg/ml each, and the incubation was continued for an additional 4 hours. The reaction mixture was precipitated sequentially with 25 and 75% ethanol in the presence of 10 mM CaCl₂.

The 75% ethanol precipitate was pelleted by centrifugation at 12,000.times.g for 30 minutes, or at a lower rpm for a longer time. The supernatant was transferred to dialysis tubing. The reaction mixture was filtered through a 0.45 μm pore-size membrane and precipitated sequentially with 25 and 75% ethanol in the presence of 10 mM CaCl₂. The 75% ethanol precipitate was dialyzed extensively against water at 3 to 8° C. and freeze-dried. The powder was dissolved in 0.2 M NaCl/0.05 M Tris HCl, pH 7.0. The resulting crude material was loaded onto a Q Sepharose column in 0.2 M NaCl/0.05 M Tris HCl, pH 7.0, and eluted with a 0.2-0.4 M NaCl linear gradient. Fractions that contained antigen, as detected by capillary precipitation with antiserum from Example 2, were pooled, dialyzed, and freeze-dried. Most of the antigen eluted at 0.32-0.35 M NaCl/0.05 M Tris HCl.

The crude antigen thus obtained was treated with 1 mg lysozyme per 10 mg crude antigen in 10 mM CaCl₂ to digest residual peptidoglycan contamination. The lysozyme-treated crude antigen then was further purified on a Sephacryl S-300 gel filtration column in 0.2 M NaCl/PBS 1× to obtain substantially pure antigen. All reactive material was screened using whole antiserum.

### EXAMPLE 3

### Characterization of Antigen

Chemical and physicochemical analysis of purified antigen. Purified 336PS showed Kd on Superose 12 HR of 0.30-0.36. The antigen itself was almost free of protein, but typically is found in combination with about 3-18% peptidoglycan, less than 1% nucleic acids, and contains about 5% phosphorus. No 0-acetyl groups were detected by colorimetric assay (Hestrin (1949) Biol. Chem. 189:249). Immunoelectrophoresis of purified antigen and elution pattern on ion-exchange column during purification process indicate a negatively-charged molecule.

Analysis of the carbohydrate composition of the antigen by HPAEC (high pH anion exchange chromatography) after its adequate complete hydrolysis showed that it is composed of N-acetyl-glucosamine and ribitol, typically in about a 1:1 ratio. A phosphorus assay indicated the presence of phosphorus as a phosphodiester function, clarifying the origin of the negative charge. The composition of this phosphorylated polymer is the same as that of the known S. aureus teichoic acid (from S. aureus Wood strain). Indeed, a comparison of the proton nuclear magnetic resonance spectra of this teichoic acid and 336PS showed a strong similarity between the two structures, but it also revealed a major difference in the chemical shifts of their respective single anomeric proton (4.75 ppm in 336PS versus 4.87 ppm in teichoic acid). The comparison of the ¹³C-nuclear magnetic resonance spectra of the two compounds confirms this difference. Analysis of the ¹H—¹H homonuclear correlation (COSY) and the ¹H—¹³C heteronuclear multiple quantum correlation (HMQC) nuclear magnetic resonance spectra of the antigen allowed the establishment of its structure without ambiguity. The antigen comprises a 1,5-poly(ribitol phosphate) polymer chain in which the 3-position of the ribitol is substituted by N-acetyl-β-D-glucosaminyl residues.

Both S. aureus 336PS and S. aureus teichoic acid were subjected to periodate oxidation treatment. Unlike 336PS, S. aureus teichoic acid was severely degraded upon periodate oxidation, clearly indicating a critical structural distinction between the two.

Structural analysis of purified polysaccharide. ¹H NMR and ¹³C NMR spectroscopy confirmed the presence of one glycoside, as indicated by the presence of one anomeric signal at 4.75 ppm and 102.4 ppm respectively. This confirms the presence of monosaccharide as a component. The large value of J_{H1,H2} (8.98 Hz) demonstrated that this residue is in the β-configuration. Signals at 23.2 ppm (NAc-methyl) in ¹H NMR and 175 ppm (NAc-carbonyl) in ¹³C NMR spectrum suggested that it was N-acetylated.

The mobility of purified antigen in immunoelectrophoresis (IEF) indicates the presence of negatively-charged groups. The purified antigen does not contain neutral sugars as detected by the phenol sulfuric assay. The K_{d} of purified antigen was 0.34 on Superose 12 HR column, which is a smaller molecular size material in comparison with Type 5 (K_{d} of 0.017), Type 8 (K_{d} of 0.061) and teichoic acid (K_{d} of 0.18).

Immunochemical analysis of S. aureus 336PS. Purified 336PS reacted with a single precipitin band with whole cell antisera to the prototype 336 strain in a double immunodiffusion assay, while teichoic acid isolated from S. aureus Wood strain or S. epidermidis (ATCC 55254) did not cross-react with the antiserum raised against the prototype strain in this assay.

### EXAMPLE 4

### Preparation of Antigen-Immunocarrier Conjugates

Immunization of ICR mice with purified polysaccharide induced no detectable antibody response by ELISA. To increase immunogenicity of the polysaccharide, S. aureus 336PS was conjugated to a recombinantly-produced, non-toxic Pseudomonas aeruginosa exotoxin A (rEPA) using adipic acid dihydrazide (ADH) as the linker. CNBr-activated 336PS was covalently bound to the protein using adipic acid dihydrazide as the linker. Carbodiimide was employed to bind a linker to protein carboxyls. Resultant conjugate was purified further to separate it from unconjugated reactants and reagents. Conjugate was characterized for 336PS to protein ratio, size, and the amount of unconjugated 336PS, if any, and then was formulated in saline or other suitable diluent for immunogenicity testing.

### EXAMPLE 5

### Immunogenicity of S. aureus 336 Conjugate Vaccine

The 336PS conjugate vaccine was injected into ICR mice three times in two weeks intervals. Immune response to 336PS was tested one week after each injection. Results showed that three injections were needed to elicit a significant rise in 336PS antibodies. Conjugated 336PS also was used to generate hyperimmune 336PS specific antisera.

Rabbit antibodies from rabbit immunized with 336PS conjugate vaccine formed a precipitin line with both S. aureus 336PS and teichoic acid from S. aureus Wood strain and also S. epidermidis (ATCC 55254). This indicates that 336PS conjugate vaccine generates cross-reactive antibodies with polysaccharides isolated from other staphylococcal species.

The 336PS conjugate vaccine was injected into rabbits with adjuvant (CFA followed by IFA) at a 1:1 ratio. Positive bleeds were combined and IgGs were purified on a protein G column. Conjugate-raised IgG and S. aureus 336 whole cell IgG recognized 336PS as an identical antigen in an immunodiffusion assay against the antigen. Purified anti-conjugate serum IgG was shown to contain 12.2 mg/ml total IgG by a 280 nm UV scan and 0.7 mg/ml antigen-specific IgG by ELISA. Whole cell antiserum, anti-whole cell IgG, and anti-conjugate IgG were used in opsonophagocytosis assays and animal models.

### EXAMPLE 6

### In vitro Opsonophagocytic Activity of S. aureus 336 Conjugate Vaccine in Homologous and Non-Homologous Strains of S. aureus

Frozen beads of S. aureus 336 strains were inoculated in 5 ml of Columbia MgCl₂/CaCl₂ broth and were incubated at 37° C. with 200 rpm for 16 hours. Cells were adjusted in saline to 0.02 O.D. at 540 nm to yield an approximate concentration of 2×10⁶ CFU/ml. Meanwhile, freshly prepared HL-60 cells that had prior been induced by dimethyl sulfoxide (DMSO) were spun at 1200 rpm. The pelleted cells were resuspended in 1 ml opsonization media (1× MEM [Minimum Essential Medium, with Earle's salt, w/o glutamate], supplemented with 0.1% gelatin) to yield a cell concentration of 1×10⁷ cells/ml. Simultaneously, human complement (plasma) was prepared by diluting human plasma to 1:80 dilution in opsonization media.

To initiate the assay, 50 μl of bacterial suspension, 50 μl of diluted complement, 50 μl of the induced HL-60 cells suspension and 50 μl of buffer or diluted rabbit antibodies (normal rabbit serum, 336 whole cell antiserum, Wood whole cell antiserum, and 336PS conjugate antiserum) were added per individual wells of polystyrene round bottom microtiter plates (Corning Glass Works). After mixing, a 25 μl aliquot was taken and plated on Tryptic Soy Agar (TSA) plates at 1:10, 1:100, 1:500, 1:1,000 and 1:2,000 dilution in distilled water for 0 time measurement. Simultaneously the reaction plate was spun at 37° C. for 5 minutes at 1200 rpm and was incubated for an hour at 37 C in 5% CO₂ atmosphere. At time 1 hour, samples were plated in the same fashion as at the 0 hour time point. The TSA plates were incubated for 16-24 hours and the emerging colonies were enumerated and used to calculate percent survival by following formula: [CFU/mL (1 hour counts)/CFU/ml (Time 0 counts)]×100.

The capability of conjugate-raised antibodies to mediate opsonophagocytic killing of multiple S. aureus strains specifically serotyped as being Type 336 was evaluated on randomly selected isolates and vancomycin-intermediate S. aureus 14358. The results showed that 336PS conjugate elicited antibodies that mediated opsonophagocytic killing of these isolates, with more than 80% reduction of bacterial cell counts.

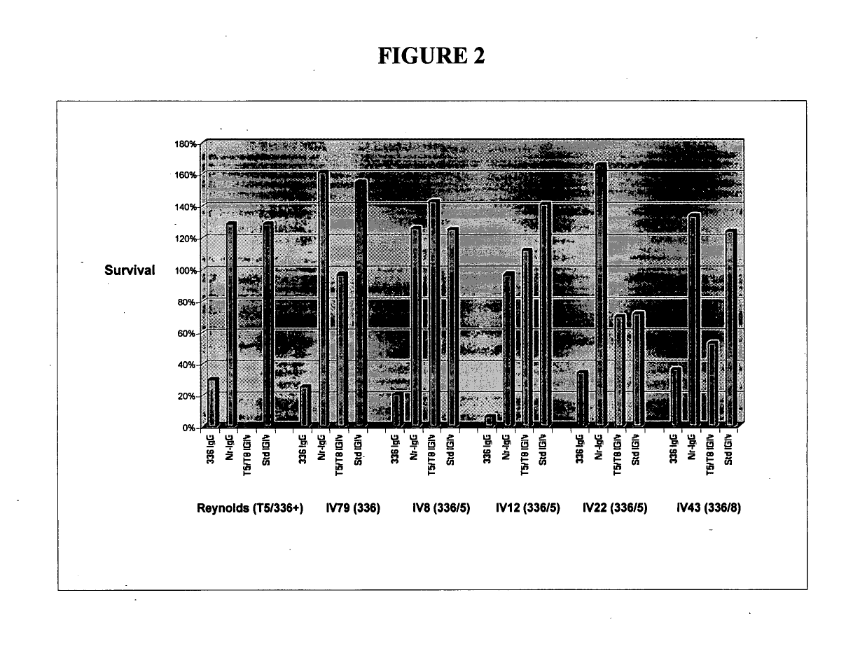

The 336PS conjugate-raised antibodies were tested for the ability to mediate opsonophagocytic killing of S. aureus Type 336/5 and Type 336/8 strains. Opsonic killing of mixed serotype S. aureus isolates by purified 336PS conjugate rabbit IgG (“336-IgG”) is shown in FIG. 2. 336-IgG was able to mediate opsonophagocytosis of serotype 336, 336/5 and 336/8 strains. As controls, normal rabbit IgG (Nr-IgG), T5 congugate human hyperimmune IgG (T5/T8 IGIV) and purified standard human IgG (Std IGIV) were also evaluated for opsonization of S. aureus isolates.

The role of 336PS-specific antibodies in opsonophagocytic killing of S. aureus Type 336 was evaluated by absorption with free 336PS and S. aureus teichoic acid. Samples of antibodies (normal rabbit serum, 336 whole cell antiserum, and 336PS conjugate antiserum) were absorbed by overnight incubation with S. aureus 336PS and Wood teichoic acid at 4° C. The mixtures were clarified by microcentrifugation and the resulting supernatants were evaluated for opsonic activity. Opsonophagocytic killing was inhibited by preincubation of antibodies with native 336 polysaccharide, but not with teichoic acid from Wood strain. This confirmed the importance of the structural difference between S. aureus 336PS and S. aureus teichoic acid and subsequently the role of 336PS specific antibodies in killing of homologous bacterial serotype.

### EXAMPLE 7

### Cross-Reactivity of Antibodies Raised Against 336PS Conjugate or 336 Whole Cell Vaccine and Conjugate with S. epidermidis Antigen and S. aureus Teichoic Acid

The cross-reactivity of 336PS antibodies with S. epidermidis polysaccharide (PS1) antigen and S. aureus teichoic acid (SA TA) was measured using an inhibition-ELISA. Both anti-336 whole cell (WC) rabbit serum and murine antiserum raised against 336PS conjugate vaccine were evaluated.

Tested antiserum (anti-336PS conjugate or anti-336 whole cell) was diluted in PBB (1% BSA, 0.3% Brij in 1× PBS) to achieve a concentration that is double the concentration that gives an OD₄₅₀ of ˜2.0. S. aureus 336PS or S. epidermidis polysaccharide (PS1) as disclosed in U.S. Pat. No. 5,866,140 was 2-fold serially diluted and 200 μl of each dilution in separate Eppendorf tubes were mixed with 200 μl of antiserum or IgG. The antiserum/inhibitor mixtures were incubated at 37° C. for one hour and tested using ELISA procedure as follows.

Microplates were coated with 100 μl/well of 4 μg/ml solution of either SA 336PS or SE PS1 or SA TA in PBS from columns 2-12 and incubated overnight at room temperature. Plates were aspirated and blocked with 200 μl/well of 1% BSA in PBS for 1 h at 37° C. Plates were washed 5 times with 0.9% NaCl containing 0.1% Brij. To each well from column 2-12 and the rows B-H, 100 μl PBB was added. Wells A2 and A3 received 200 μl of antiserum 2-fold diluted with PBS to reach OD₄₅₀ 2.0 (no inhibitor) and the rest of the wells in row A received 200 μl of the pre-incubated serum/inhibitor mixtures. Specimens in A2-A12 were 2-fold serially diluted down to H2-H12. Plates were incubated for 1 hour at 37° C., then washed and filled with 100 μl/well of goat horse-radish peroxidase anti-rabbit IgG (Fc) of relevant animal species. Plates were incubated for 1 hour at 37° C., washed and filled with 100 μl/well of H₂O₂/TMB substrate. The reaction was developed for 10 minutes and then stopped with 100 μl/well of 1M phosphoric acid. Intensity of the color developed in the wells was monitored at 450 nm using a microplate reader.

Table 1A shows the inhibition of binding 336 conjugate antiserum or 336 whole whole cell antiserum to 336PS (coating antigen) with homologous PS inhibitor SA 336PS (from S. aureus 336) and heterologous PS inhibitors SE PS1 (from S. epidermidis ATCC 55254) and SA TA (from S. aureus Wood strain). Table 1B shows inhibition of binding 336PS conjugate antiserum to different coating antigens (SA 336PS, SE PS1 or SA TA) with homologous and heterologous PS inhibitors. The term “homologous” here refers to 336PS because each antiserum (anti-WC or anti-conjugate) was raised against either Type 336 bacterium or Type 336 derived PS. SE PS1 and SA TA are in this sense “heterologous” polysaccharides.

TABLE 1A

| | Inhibitors, and their concentration (μg/ml) | | | |
|---|---|---|---|---|
| Coating antigen | conferring 50% inhibition of serum binding | | | |
| 336PS | SA 336PS | SE PS1 (ATCC 55254) | | SA TA |
| 336PS-conjugate | 0.51 (1) | 2.5 | (5) | 28 (60) |
| antiserum (Ratio) | | | | |
| 336-whole cell | 17 (1) | NA | (∞) | 197 (12) |
| antiserum (Ratio) | | | | |

NA: 50% inhibition was not reached at the highest tested inhibitor concentration of 250 μg/ml
Ratio stands for the ratio of the concentrations of heterologous inhibitor to homologous inhibitor
“336PS” needed to render 50% inhibition of binding antiserum to coating antigen.
∞ shows that it could not be estimated since 50% inhibition was not reached

TABLE 1B

| | Inhibitors, and their concentration (μg/ml) | | |
|---|---|---|---|
| 336PS-conjugate | conferring 50% inhibition of serum binding | | |
| antiserum | SA 336PS | SE PS1 (ATCC 55254) | SA TA |
| Antigen: SA | 0.51 (1) | 2.5 (5) | 28 (60) |
| 336PS (Ratio) | | | |
| Antigen: SE | 0.15 (1) | 0.5 (3) | 7 (47) |
| PS1 (Ratio) | | | |
| Antigen: SA | 0.25 (1) | 1.3 (5) | 3 (12) |
| TA (Ratio) | | | |

Results in Table 1A suggest that 336 conjugate antiserum contains antibodies to 336 PS that can cross-react with SE PS1 and SA TA. The ratios of 50% inhibitor concentrations of heterologous to homologous inhibitor reflect comparative cross-reactivity powers by heterologous versus homologous PS to 336 conjugate antiserum. SE PS1 and SA TA are about 5 and 12 times, respectively, weaker inhibitors than 336PS of binding 336PS conjugate antiserum to 336PS. SA 336PS is also the strongest inhibitor of 336 whole cell antiserum. PS1 does not confer a 50% inhibition of 336 WC antiserum, indicating a very low cross-reactivity, if any, of PS1 with 336 WC antiserum. Table 1B compares inhibition powers of heterologous polysaccharides (SE PS1 and SA TA) and 336 PS towards binding 336PS-conjugate antiserum with either 336PS, SE PS1 or SA TA. It is shown that 336PS is the best inhibitor of anti-336PS conjugate antiserum regardless to what polysaccharide this antiserum binds. These results confirm that 336PS conjugate elicits antibodies that carry high specificity to 336PS, yet also cross-react with other antigens due to shared similarities of some antigenic determinants.

### EXAMPLE 8

### Efficacy of 336 Conjugate-Derived Antibodies in Clearing S. epidermidis Bacteremia

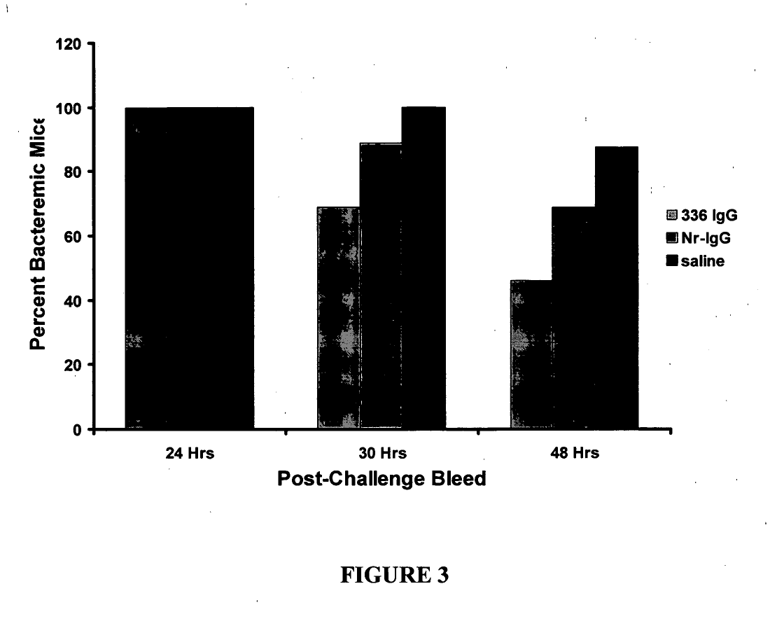

The ability of 336PS conjugate to clear Staphylococcal bacteremia was assessed. ICR mice were passively immunized SQ with purified rabbit 336-rEPA conjugate derived immunoglobulin or with purified rabbit PS1-rEPA conjugate derived immunoglobulin. Twenty-four hours later mice were challenged intraperitoneally at 5×10⁷ CFU/ 5% mucin-saline with a S. epidennidis prototype strain that expresses S. epidermidis PS1. At 24 hours, 30 hours and 48 hours post-challenge, 10 mice per group were exsanguinated, and blood samples were streaked onto tryptic soy agar (TSA) agar plates for S. epidermidis blood cultures. Data from this study demonstrated that S. epidermidis bacteremia was cleared by 336PS conjugate vaccine derived IgGs, indicating that 336 conjugate-derived immunoglobulin efficiently cross-clears S. epidermidis bacteremia. The results are shown in FIGS. 3A and 3B.

### EXAMPLE 9

### Efficacy of 336PS Monoclonal Antibodies in S. aureus Lethal Challenge

BALB/c mice were immunized s.c. with 500 μg of appropriate 336 monoclonal antibody 48 hours prior to challenge. On following day, mice were intraperitoneally primed with phosphate buffered saline and challenged the next day with different S. aureus 336 prototype isolate. The monoclonal antibodies provided specific protection against S. aureus challenge. The results are shown in Table 2.

TABLE 2

| Immunization | | | | | |
|---|---|---|---|---|---|
| w/MAb (500 μg | Bacterial | Post-Challenge Survival | | | |
| Dose s.c.) | Challenge (IP) | (Percent Survival) | | | |
| (Day −2) | (Day 0) | 24 | 40 | 5-7 Days | |
| S. aureus 336-119 | ˜2.5 × 10⁵ | 19/28 | 19/28 | 19/28 | (67.8%) |
| S. aureus 336-560 | CFU/500 μL of | 25/28 | 25/28 | 25/28 | (89.3%) |
| E. coli 400 | S. aureus 336, | 3/28 | 3/28 | 3/28 | (10.7%) |
| PBS | 5% Hog Mucin/PBS. | 0/28 | 0/28 | 0/28 | (0%) |
| | Serotype: 336 | | | | |

### EXAMPLE 10

### Protective Efficacy of 336PS Conjugate Vaccine in Type 336/5 and Type 336 Lethal Challenge

BALB/c mice were immunized s.c. with 2.5 μg of either Type 5 or Type 336 vaccine and adjuvant on days 0, 14, 28 and 42. On day 48, the mice were intraperitoneally primed with phosphate buffered saline and challenged the next day with 2×10⁵ CFU of either S. aureus 14538 or S. aureus 5836, which are S. aureus 336 vancomycin intermediate resistant isolates (VISA) that express 336 antigen. The former strain is serotype 336, whereas the latter is a mixed 336/T5 strain. Challenged mice were monitored for morbidity and mortality at 24 hours, 40 hours and 5-7 days after bacterial challenge.

At the conclusion of the study, mice that had been immunized with 336PS conjugate vaccine showed 100% protection against both the challenge isolates. The results are shown in Table 3.

TABLE 3

| | | | Post-Challenge | | |
|---|---|---|---|---|---|
| s.c. Immunization | | Bacterial | Survival | | |
| | (Day 28 and 42) | Challenge | (percent Survival) | | |
| (Day 0 | (2.5 μg vaccine + | (IP) | | | 5-7 |
| and 14) | 100 μg adjuvant) | (Day 49) | 24 | 40 | Days |
| S. aureus | S. aureus 336PS | ˜1 × 10⁵ CFU | 14/14 | 14/14 | 14/14 |
| 336 | conjugate | S. aureus 14358/ | | | (100%) |
| PBS | PBS + adjuvant | 4% Mucin-PBS | 4/10 | 4/10 | 3/10 |
| | | Serotype: 336 | | | (30%) |
| S. aureus | S. aureus 336 | 1 × 10⁵ CFU | 15/15 | 15/15 | 15/15 |
| 336 | conjugate | S. aureus 5836/ | | | (100%) |
| PBS | PBS | 4% Mucin-PBS. | 4/10 | 4/10 | 4/10 |
| | | Serotype: 336/T5 | | | (40%) |

Thus, a method of preventing or treating bacterial infection in an individual has been described according to the present invention. Many modifications and variations may be made to the techniques and structures described and illustrated herein without departing from the spirit and scope of the invention. Accordingly, it should be understood that the methods described herein are illustrative only and are not limiting upon the scope of the invention.

What is claimed is:

1. A method for preventing infection in a population of patients at risk for infection by various species of Staphylococcus or various types of Staphylococcus aureus, comprising administering to a patient in the population a composition comprising a conjugate of an isolated S. aureus antigen that contains N-acetylglucosamime linked to ribitol, wherein the antigen binds with antibodies to S. aureus Type 336 deposited under ATCC 55804, wherein the conjugate of the isolated S. aureus antigen produces antibodies that protect against a species or type of Staphylococcus other than S. aureus Type 336.

2. A method according to claim 1, wherein the antigen comprises a 1,5-poly(ribitol phosphate) polymer chain in which the 3-position of the ribitol is substituted by N-acetyl-β-D-glucosaminyl residues.

3. A method for preventing infection in a population of patients at risk for infection by Staphylococcus epidermidis, comprising administering to a patient in said population a composition comprising a conjugate of an isolated S. aureus antigen that contains N-acetylglucosamime linked to ribitol, wherein the antigen binds with antibodies to S. aureus Type 336 deposited under ATCC 55804, wherein the isolated S. aureus antigen produces antibodies that protect against S. epidermidis.

4. A method according to claim 3, wherein the antigen comprises a 1,5-poly(ribitol phosphate) polymer chain in which the 3-position of the ribitol is substituted by N-acetyl-β-D-glucosaminyl residues.

5. A method for treating infection in a population of patients at risk for developing infection by various species of Staphylococcus or various types of Staphylococcus aureus, comprising administering to a patient in said population a composition comprising antibodies to a conjugate of an isolated S. aureus antigen that contains N-acetylglucosamime linked to ribitol, and that binds with antibodies to S. aureus Type 336 deposited under ATCC 55804.

6. A method for treating infection in a patient diagnosed as having a S. epidermidis infection, comprising administering to the patient a composition comprising antibodies to a conjugate of an isolated S. aureus antigen that contains N-acetylglucosamime linked to ribitol, and that binds with antibodies to S. aureus Type 336 deposited under ATCC 55804.

7. A method according to claim 5, wherein said composition comprises a monoclonal antibodies.

8. A method according to claim 6, wherein said composition comprises a monoclonal antibodies.

9. A method according to claim 5, wherein said composition comprises polyclonal antibodies.

10. A method according to claim 6, wherein said composition comprises polyclonal antibodies.
